(19) United States
(12) Patent Application Publication
(10) Pub. No.: US 2008/0280996 A1
(43) Pub. Date: Nov. 13, 2008

(54) Use of Carophyllenes in the Manufacture of Medicaments and Treatment of Bodily Conditions of Inflammation and Inflammatory Pain

(76) Inventors: Luiz F. Pianowski, Atibaia-SP (BR); Joao B. Calixto, Florianopolis-SC (BR); Dagoberto de C. Brandao, Sao Paulo-SP (BR)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(21) Appl. No.: 11/576,351

(22) PCT Filed: Oct. 1, 2004
(86) PCT No.: PCT/BR04/00189

§ 371 Date: Mar. 6, 2008

(51) Int. Cl. A61K 31/015; C07C 13/271; C07C 13/47; A61P 29/00

(52) U.S. Cl. 514/763; 585/16

(57) ABSTRACT

The invention concerns the use of caryophyllenes related to medicaments and to the treatment of bodily conditions of inflammation and inflammatory pain.

### FIELD OF THE INVENTION

The present invention concerns the use of caryophyllenes related to medicaments and to the treatment of bodily conditions of inflammation and inflammatory pain. It relates particularly to the use of caryophyllenes in the manufacture of medicaments for the treatment of inflammatory conditions of the animal body, including the human body. The invention also concerns the use of caryophyllenes for the treatment of inflammatory conditions of the body, including inflammatory pain.

### BACKGROUND OF THE INVENTION

Caryophyllenes are known chemical compounds, useful in various applications. For instance patent document U.S. Pat. No. 3,987,008 reveals sesquiterpenic derivatives as odor and taste-modifying agents; In J. Nat. Prod. 1992 July; 55(7):999-1003, beta-caryophyllene and alpha-humulene as cited as potential anticarcinogenic agents; in patent application WO alpha and beta-humulene and (−)-beta-caryophyllene are cited in the control of whitefly species; in U.S. Pat. No. 5,314,693 alpha-humulene is cited as a repellent for pine wood nematodes; in patent application WO 02078719 alpha and beta-caryophyllene are comprised in antitumor compositions.

### SUMMARY OF THE INVENTION

The present invention concerns, in one particular aspect, a new and useful use for caryophyllenes, more particularly alpha-humulene or beta-caryophyllene, as anti-inflammatory and as analgesic, in a broad sense. In a more specific sense, the compounds were found to be useful inhibitors of entities that are known to be involved in the inflammatory process:

pro-inflammatory cytokines IL-1β (interleukin 1β) and TNFα (tumor necrosis factor α);

PGE2 (prostaglandin-E2),

Expression of COX-2 (cycloxigenase-2) and iNOS (inducible nitric oxide synthase) enzymes

One particular example of caryophyllene of the invention is alpha-humulene, a sesquiterpene identified with the CAS (Chemical Abstracts Service) registry number 6753-98-6, also known as alpha-caryophyllene, represented by the following structure:

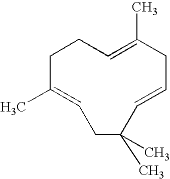

Another particular example of caryophyllene of the invention is the trans-caryophyllene (or beta-caryophyllene), also a sesquiterpene, identified with the CAS (Chemical Abstracts Service) registry number 87-44-5, represented by the following alternative structures A and B:

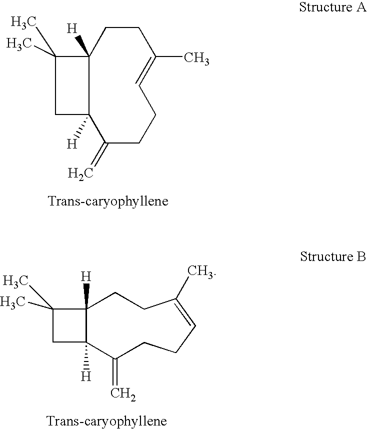

According to the meaning employed herein, mention to caryophyllenes, object of the invention, includes the molecules as such, their salts, isomers, metabolites, pro-drugs, solvates (including hydrates) and adducts.

Terpenes, including sesquiterpenes, are often mentioned as components of complex mixtures extracted from plants, where—as known to a persons skilled in the art—it is undetermined what compound or compounds are effective, how much effective they are, and whether they are active by themselves, by way of the vehicle/solvent the composition contains (water, alcohol, other solvents, mixtures of those, etc), or by way of their interaction with other components within the mixtures. Individual terpenes per se, of natural origin or products of synthesis (for instance J. Am. Chem. Soc., 99, 3864 (1977)), are rarely mentioned as effective pharmaceutical agents. Alpha-humulene has even been mentioned to be virtually inactive concerning anti-inflammatory or chemo-therapic effects (reference: Carcinogenesis (2002), 23(5), 795-802).

The applicant has now found out that caryophyllenes, particularly alpha-humulene and trans-caryophyllene, have marked anti-inflammatory effects, including inflammatory pain, comprised therein the inhibitory effect upon the production of pro-inflammatory cytokines IL-1β and TNFα, prostaglandin PGE2, or the expression of enzymes COX-2 and iNOS.

The applicant has also found out that caryophyllenes, particularly alpha-humulene and trans-caryophyllene, have anti-allergic, particularly anti-histaminic effects.

The caryophyllenes of the invention are part of the ongoing search for drugs with direct of indirect inflammatory activity, which inhibit the physiopathology processes involved in inflammation. They are used in the control of chronic-degenerative diseases as rheumatoid arthritis, osteoarthritis, systemic lupus eritematosus, ulcerative colitis, psoriasis, atopic eczema, atherosclerosis, and other non-degenerative diseases as depression, and cellulites, and allergies.

Therefore, one of the objects of the present invention is the use of caryophyllenes, particularly alpha-humulene and/or trans-caryophyllene, or compositions comprising caryophyllenes, in the manufacture of a medicament for the treatment of inflammatory conditions of the animal body, particularly the human body.

Another object of the present invention is the use of caryophyllenes, particularly alpha-humulene and trans-caryophyllene, or compositions containing caryophyllenes, in the treatment of inflammatory conditions of the animal body, particularly the human body.

Another object of the present invention is a method of treatment of an inflammatory condition of the animal body, particularly the human body, comprising the administration of a therapeutically effective amount of caryophyllenes, particularly alpha-humulene or trans-caryophyllene, to a patient.

Another object of the present invention is the use of caryophyllenes, particularly alpha-humulene and trans-caryophyllene, or compositions containing caryophyllenes, for the inhibition of the bodily production of one or more of cytokine IL-1β, cytokine TNFα, prostaglandin PGE2, expression of enzymes COX-2 and iNOS.

### DETAILED DESCRIPTION OF THE PREFERRED EMBODIMENT

The caryophyllene of the invention, as well as compositions comprising the caryophyllene according to the invention, can be administered to the subject in need of treatment in any adequate way, enteral or parenteral, including oral, topical, transdermal, subcutaneous, intraperitonial, intravenous, by infiltration, by inhalation, transdermal, transmucosal, intramuscular, intrapulmonary, vaginal, rectal, intraocular, and sublingual. Particularly adequate ways of administration in the present invention are systemically (infiltration, oral, inhalation by spray, transdermal) and topically. The caryophyllene of the invention can be comprised in a slow or controlled release composition. Known adjuvants and excipients can be utilized in the compositions. A reference for pharmaceutical dosage forms useful for the compositions related to the inventions can be found in the publication Remington's Pharmaceutical Sciences, Mack Publishing.

The compositions comprising caryophyllene can be administered to patients as solids, liquids or semi-liquids, tablets, capsules, pills, powder, granules, suspensions, emulsions, dispersions and any other useful known form.

The compositions might contain further active agents, for instance antibiotics, depending on the desired effect.

For oral administration as tablets or capsules (both soft and hard capsules), the caryophyllene can be combined with pharmaceutically acceptable inert vehicles, such as lactose, starch, sucrose, glucose, methyl cellulose, magnesium stearate, dicalcium phosphate, calcium phosphate, manitol, sorbitol, and similars; for oral administration in the liquid form, the caryophyllenes can be combined with ethanol, glycerol, water, and similars. When desired or necessary, agglomerating agents, lubricant agents, disintegrating agents, color and fragrance can be added to the mixture. Common agglomerating agents are glucose, β-lactose, corn sweeteners, natural or synthetic gums such as gum arabica, tragacanth or sodium alginate, carboxymethylcellulose, polyethylene glycol, wax and similars. Lubricants include sodium oleate, sodium stearate, magnesium stearate, sodium benzoate, sodium acetate, sodium chloride. Disintegrants include starch, methyl cellulose, agar, bentonite, xanthan gum, and similars.

The compositions concerned in the invention can also be administrated as liposomes or coupled with soluble polymers as vehicles.

Liquid dosage forms for oral administration may comprise colorants and edulcorants to increase acceptance by patients. Acceptable vehicles for water dosage forms are, water, an appropriate oil, a saline solution, aqueous dextrose, other sugar solutions and glycols as propylene glycol or polyethylene glycols, phosphate buffer.

Compositions related to the present invention typically comprise about 1 mg to about 1000 mg of one or more caryophyllenes, particularly about 10 to 200 mg and more particularly about 30 to 100 mg. In such compositions the caryophyllene represents about 0.1 to 99% in weight, particularly about 1 to 70% and more particularly about 10 to 40%, optionally comprising at least one pharmaceutically acceptable vehicle.

### EXAMPLES

The examples that follow represent particular embodiments of the invention, and do not impose any limitation to its extension, which is limited only by the claims attached hereto.

### Example 1

### Inflammatory Nociception Induced by Carregenin

The evaluation methodology used in this test is described by Vaz et al. in J. Pharmacol. Exp. Ther. 278:304-312, 1996.

Male mice (25-35 g) were systemically (orally) treated with alpha-humulene, 50 mg/kg, administered 1 hour before the experiment. Animals treated with 0.9% saline solution (0.1 ml/10 g) were used as the control. Another group of animals was treated with paracetamol (600 mg/kg, orally, administered 1 h before the treatment), that was used as positive control. For the induction of inflammatory pain, the animals received an intraplantar injection of 0.05 ml of carrageenin (300 μg per paw) at the plantar surface of the right hind paw. This dosage causes oedema, nociception and substantial swelling of the injected paw.

The nociception was evaluated with a Von Frey filament (0.4 g) after 3, 4 and 6 hours. To obtain a basal response, the animals were pre-tested the previous day with the 0.4 g von Frey filament. Only animals with a response of about 20% were selected. The filament was applied to the right hind paw, complying with the criteria of (1) the application was perpendicular to the plantar surface, with enough pressure to cause the filament to bend, thus obtaining total pressure; (2) the animals were evaluated when the four paws were touching the screen; (3) the paw withdrawal response was considered when the animal removed the paw entirely from the support screen; (4) each animal was stimulated 10 consecutive times, each stimulation lasting 1 second; (5) each paw withdrawal event was considered as 10% of the response, with 10 withdrawal events corresponding to 100% response.

Graph 1 below compares the pain inhibition obtained by alpha-humulene with the administration of paracetamol. Each point represents the average of 5 animals, and the vertical bars the mean standard error deviation.

The graph clearly shows that, according to the invention, a caryophyllene as alpha-humulene reduced the inflammatory nociception, as a result of reduction of inflammation, as much as a known analgesic, paracetamol.

### Example 2

### Carrageenin Oedema in Mouse Paw

The test used below is described by Cunha et. al. in the publication Life Sci. 70:159-169, 2001.

Male 25 g-35 g mice were slightly sedated with ether and were injected 50 μl saline containing carrageenin (300 μl/paw) in the right paw. The left paw received the same volume of saline and was taken as a negative control. The swelling was measured with a plethysmometer (manufacturer: Ugo Basile, Italy) along various time intervals after the injection of the phlogistic agent. The difference between the volumes of the right and the left paw were quantified (in ml) and taken as an index of oedema. One hour before the test the animals were systemically treated (orally) with 50 mg/kg of alphahumulene or trans-caryophyllene.

Graph 2 below compares the inhibition of the volume of the oedema by administration of either alpha-humulene or trans-caryophyllene with inhibition obtained with the administration of dexamethasone (0.5 mg/kg, injected subcutaneously 4 h before test) and used as positive control. Oedema volume measurement time point intervals were 30, 60, 120 and 240 min, 24 h and 48 h. Each point represents the average of 5 animals, and the vertical bars the mean standard error deviation.

The graph clearly shows that, according to the invention, a caryophyllene as alpha-humulene reduced the inflammatory volume, as did dexamethasone.

### Example 3

### Bradykinin Oedema in Mouse Paw

The test used below is described by Cunha et. al. in the publication Life Sci. 70:159-169, 2001.

Male 25 g-35 g mice were slightly sedated with ether and were injected 50 μl saline containing bradykinin (BK, 3 nmol/paw), intraplantar, in the right paw. The left paw received the same volume of saline and was taken as negative control. The swelling was measured with a plethysmometer (manufacturer: Ugo Basile, Italy) along various time points intervals after the injection of the phlogistic agent. The difference between the volumes of the right and the left paw were quantified (in ml) and taken as an index of oedema. One hour before the test the animals were systemically treated (orally) with 50 mg/kg of alpha-humulene or with trans-caryophyllene.

The animals were pre-treated with 5 mg/kg of captopril, injected subcutaneously, 1 hour before the test, in order to avoid degradation of kinines.

Graphs 3A and 3B below compare the inhibition of the volume of the oedema by administration of alpha-humulene (3A) or trans-caryophyllene (3B). Oedema volume measurement time intervals were 10, 20, 30, 60, and 120 min, 24 h and 48 h. Each point represents the average of 5 animals, and the vertical bars the mean standard error deviation.

The graph clearly shows that, according to the invention, a caryophyllene as alpha-humulene or trans-caryophyllene markedly reduced bradykinin-induced paw oedema.

### Example 4

### Histamine Oedema in Mouse Paw

The test used below is described by Cunha et. al. in the publication Life Sci. 70:159-169, 2001.

Male 25 g-35 g mice were slightly sedated with ether and were injected 50 μl saline containing histamine (100 nmol/paw), intraplantar, in the right paw. The left paw received the same volume of saline and was taken as negative control. The swelling was measured with a plethysmometer (manufacturer: Ugo Basile, Italy) along various time intervals after the injection of the phlogistic agent. The difference between the volumes of the right and the left paw were quantified (in ml) and taken as an index of oedema. One hour before the test the animals were systemically treated (orally) with 50 mg/kg of alpha-humulene.

Graph 4 below compares the inhibition of the volume of the oedema by administration of alpha-humulene. Oedema volume measurement time intervals were 10, 20, 30, 60, and 120 min, 24 h and 48 h. Each point represents the average of 5 animals, and the vertical bars the mean standard deviation.

The graph clearly shows that, according to the invention, a caryophyllene as alpha-humulene significantly reduced histamine-induced oedema formation. It also indirectly shows effect against allergy.

### Example 5

### Platelet Aggregation Factor (PAF) Oedema in Mouse Paw

The test used below is described by Cunha et. al. in the publication Life Sci. 70:159-169, 2001.

Male 25 g-35 g mice were slightly sedated with ether and were injected 50 μl saline containing platelet aggregation factor (PAF, 3 nmol/paw), intraplantar, in the right paw. The left paw received the same volume of saline and was taken as negative control. The swelling was measured with a plethysmometer (manufacturer: Ugo Basile, Italy) along various time intervals after the injection of the phlogistic agent. The difference between the volumes of the right and the left paw were quantified (in ml) and taken as an index of oedema. One hour before the test the animals were systemically treated (orally) with 50 mg/kg of alpha-humulene or trans-caryophyllene.

Graph 5 below compares the inhibition of the volume of the oedema by administration of alpha-humulene (SA) and trans-caryophyllene (5B). Oedema volume measurement time intervals were 30, 45, 60, and 120 min. Each point represents the average of 5 animals, and the vertical bars the mean standard deviation.

The graph clearly shows that, according to the invention, a caryophyllene as alpha-humulene or trans-caryophyllene markedly reduced PAF-induced oedema formation. As PAF is also known to be involved in allergic processes, such data further reinforces the use of caryophyllenes in the management of allergic states.

### Example 6

### Arachidonic Acid Oedema in Mouse Ear

The ear oedema in the test below was measured according to Calixto et. al. in the publication Prostaglandins, 5: 515-526, 1991, with minor modifications.

Male 25 g-35 g mice, in a first group, were topically applied, in the inner surface of the ears, an ointment comprising a range from 0.025 to 0.2% alpha-humulene or trans-caryophyllene. In the positive control group, the animals were topically applied 0.05 mg of phenidone per ear. After 60 minutes, the animals received 20 μl of arachidonic acid (2 mg/ear), dissolved in acetone, in the inner surface of the right ear. The oedema was measured using a digital micrometer, and the responses were expressed as μm, the difference between the ear thickness before and after the application of arachidonic acid. The responses of the animals treated with caryophyllenes were compared to those observed in the control group animals, treated with base ointment.

Graphs 6A and 6B below compare the inhibition of the volume of the oedema by topic administration of alpha-humulene (6A) and trans-caryophyllene (6B). Oedema volume measurement was performed after application of 0.025%, 0.05%, 0.1, and 0.2% caryophyllene content ointment, compared to the oedema volume caused by the application of phenidone and arachidonic acid (C). Each point represents the average of 5 animals, and the vertical bars the mean standard error deviation.

The graphs clearly show that, according to the invention, the topic application of a caryophyllene as alpha-humulene or trans-caryophyllene markedly reduced oedema formation, in a dose-dependent manner.

### Example 7

### Levels of Pro-Inflammatory Cytokine IL-1B

The test used below is described by Campos et. al. in the publication Br. J. Pharmacol. 135: 1107-1114, 2002, with minor modifications.

Male 160-180 g rats were orally given 50 mg/kg of alpha-humulene. Animals treated with 0.9% (0.1 ml/10 g) saline were used as control. Another group of animals was treated with 0.5 mg/kg dexamethasone, subcutaneously, 4 hours before the test, and used as positive control. After 60 minutes, the animals received intraplantar injections of 100 μl of carrageenin (300 μg/paw) and were sacrificed after 180 minutes. Control animals received saline. The subcutaneous tissue of the injected paws was removed and put in a phosphate buffer containing 0.5% tween 20, 0.1 mM benzametonium chloride, 10 mM EDTA, 2 μg/m aprotinin, 0.1 mM PMSF (phenyl methyl sulfonyl fluoride) and 0.5% BSA (bovine serum albumin). The tissues were homogenized and centrifuged at 3000 g, for 10 min, at −4° C. The supernatant was used in the test. The levels of IL-1β were measures with an Elisa kit, according to the manufacturer's instructions (R & D Systems®, USA). The tests were performed in duplicate, and repeated three times. The answers are expressed in pg/mg of tissue.

Graph 7 below (each result represents the average of 5 animals, and the vertical bars the mean standard error deviation) compares the inhibition of production of inflammatory cytokine IL-1β induced by carrageenin in the paws of rats.

The graph clearly shows that, according to the invention, the administration of a caryophyllene, such as alpha-humulene, markedly inhibited the production of pro-inflammatory cytokine IL-1β induced by carrageenin in the paws of rats.

### Example 8

### Levels of Pro-Inflammatory Cytokine TNFα

The test used below is described by Campos et. al. in the publication Br. J. Pharmacol. 135: 1107-1114, 2002, with minor modifications.

Male 60-180 g rats were orally given 50 mg/kg of trans-caryophyllene. Animals treated with 0.9% (0.1 ml/10 g) saline were used as control. Another group of animals was treated with 0.5 mg/kg dexamethasone, subcutaneously, 4 hours before the test, and used as positive control. After 60 minutes, the animals received intraplantar injections of 100 μl of carrageenin (300 μg/paw) and were sacrificed after 180 minutes. Control animals received saline. The subcutaneous tissue of the injected paws was removed and put in a phosphate buffer containing 0.5% tween 20, 0.1 mM benzametonium chloride, 10 mM EDTA, 2 μg/m aprotinin, 0.1 mM PMSF (phenyl methyl sulfonyl fluoride) and 0.5% BSA (bovine serum albumin). The tissues were homogenized and centrifuged at 3000 g, for 10 min, at −4° C. The supernatant was used in the test. The levels of TNFαwere measures with an Elisa kit, according to the manufacturer's instructions (R & D Systems®, USA). The tests were performed in duplicate, and repeated three times. The answers are expressed in pg/mg of tissue.

Graph 8 below (each result represents the average of 5 animals, and the vertical bars the mean standard error deviation) compares the inhibition of production of inflammatory cytokine TNFα induced by carrageenin in the paws of rats.

The graph clearly shows that, according to the invention, the administration of a caryophyllene, such as trans-caryophyllene, markedly inhibited the production of the pro-inflammatory cytokine TNFα induced by carrageenin in the paws of rats.

### Example 9

### Levels of PGE2

The test used below is described by Pinheiro et. al. in the publication Inflamm. Res. 51: 603-610, 2002, with minor modifications.

Male 160-180 g rats were orally given 50 mg/kg of trans-caryophyllene. Animals treated with 0.9% (0.1 ml/10 g) saline were used as control. Another group of animals was treated with 0.5 mg/kg dexamethasone, subcutaneously, 4 hours before the test, and used as positive control. After 60 minutes, the animals received intraplantar injections of 100 μl of carrageenin (300 μg/paw) and were sacrificed after 180 minutes.

The exsudate of the paws was collected by dialysis with the help of two polyethylene canulas, and was utilized for the quantification of PGE2, with an Elisa kit, according to the manufacturer's instructions (R & D Systems®, USA). The tests were performed in duplicate, and repeated three times. The answers are expressed in pg/mg of tissue.

Graph 9 below (each result represents the average of 5 animals, and the vertical bars the mean standard error deviation) compares inhibition of the PGE2 level growth induced by carrageenin in the paws of rats, by administration of alpha-humulene and trans-caryophyllene, compared to the effect obtained by treatment with dexomethasone.

The graph clearly shows that, according to the invention, the administration of a caryophyllene, such as alpha humulene or trans-caryophyllene, markedly inhibited the growth of PGE2 levels induced by carrageenin in the paws of rats.

### Example 10

### Inhibition of the Expression of Enzymes Cox-2 and iNOS

The expression of enzymes COX-2 and iNOS were determined by Western blot according to the methodology described by Medeiros et. al. in the publication Circ Res. 28:1375-1382, 2004.

Male 160-180 g rats were orally given 50 mg/kg of trans-caryophyllene. Animals treated with 0.9% (0.1 ml/10 g) saline were used as control. Another group of animals was treated with 0.5 mg/kg dexamethasone, subcutaneously, 4 hours before the test, and used as positive control. After 60 minutes, the animals received intraplantar injections of 100 μl of carrageenin (300 μg/paw) and were sacrificed after 180 minutes, and subcutaneous paw tissue was removed 240 minutes after the carrageenin injection.

The collected tissue was immediately frozen in liquid nitrogen and re-suspended in a buffer of hypotonic lysis (10 mM HEPES N-2-hydroxyethylpiperazine-N′-2-ethanesulfonic acid, 1.5 mM MgCl2, 10 mM KCl, 0.5 mM PMSF phenylmethylsulphonyl fluoride, 1.5 μg/ml trypsin inhibitor, 7 μg/ml pepstatin A, 5 μg/ml leupeptin, 0.1 mM benzamidine 0.1 mM and 0.5 mM dithiothreitol) and homogenized. The homogenate was divided in three 2 ml aliquots, cooled in ice for 15 minutes, vigorously agitated and once again cooled in ice, in the presence of 20 μl 10% non-ionic detergent Nonidet P-40 (Roche Diagnostics, USA). The nuclear fraction was precipitated by centrifugation (1,500 g, 5 minutes) and the supernatant containing the cytosolic extract was stored at −70° C. for the Western blot tests. The protein concentration was determined by the Bradford method (BioRad Laboratories Inc. kit, Milan, Italy). The extracts were boiled with v/v equivalent amounts of Laemmly buffer (125 mM of Tris-HCl, 2 mM of EDTA, 4% of dodecyl sodium sulphate, 20% of glycerol, 10% of 2-mercaptoethanol and 0.1% of Comassie brilliant blue, pH 6.8). The proteins were transferred to nitrocellulose membranes (100 μg/well) and separated by electrophoresis. The membranes were later blocked by overnight incubation (4° C.) with skimmed powder milk (10% PBS), and then incubated with the anti-iNOS or anti-COX-2 antibodies for 1 h at room temperature. The membranes were washed three times with 10% Triton-X in PBS with the antibody peroxidase conjugated (anti-rabbit). The bands thus obtained were quantified using a chemoluminescence kit and densitometry analysis (relative units) in radiographic films.

Graph 10 below (each result represents the average of 5 animals, and the vertical bars the mean standard error deviation) compares inhibition of expression of the COX2 enzymes, obtained by the administration of alpha-humulene and trans-caryophyllene, when evaluated in the subcutaneous tissue or the paw injected with carrageenin, compared to the expression of COX2 induced by carrageenin obtained by treatment with dexomethasone.

The graph clearly shows that, according to the invention, the administration of a caryophyllene, such as alpha humulene or trans-caryophyllene, markedly inhibited the expression of enzymes COX2 induced by carrageenin in the paws of rats.

Graph 11 below (each result represents the average of 5 animals, and the vertical bars the mean standard error deviation) compares inhibition of expression of the iNOS enzymes, obtained by the administration of alpha-humulene, when evaluated in the subcutaneous tissue or the paw injected with carrageenin, compared to the expression of iNOS induced by carrageenin obtained by treatment with dexomethasone.

The graph clearly shows that, according to the invention, the administration of a caryophyllene, such as alpha humulene, markedly inhibited the expression of enzymes iNOS induced by carrageenin in the paws of rats.

The examples and the information provided herein concern particular embodiments of the present invention, which is only limited by the breath of the claims attached hereto.

1. Use of caryophyllenes, or compositions comprising caryophyllenes, characterized by the fact that it is in the manufacture of a medicament for the treatment of inflammatory conditions and inflammatory pain of the animal body, particularly the human body.

2. Use of caryophyllenes, or compositions comprising caryophyllenes, characterized by the fact that it is in the treatment of inflammatory conditions and inflammatory pain of the animal body, particularly the human body.

3. Use of caryophyllenes, or compositions comprising caryophyllenes, characterized by the fact that it is for the inhibition of the bodily production of one or more of the group comprising cytokine IL-1β, cytokine TNFα, prostaglandin PGE2, or expression of enzymes COX-2 and iNOS.

4. Use of caryophyllenes, or compositions comprising caryophyllenes, characterized by the fact that it is in the manufacture of a medicament for the inhibition of the bodily production of one or more of the group comprising cytokine IL-1β, cytokine TNFα, prostaglandin PGE2, or expression of enzymes COX-2 and iNOS.

5. Use of caryophyllenes according to one of claims 1-4 characterized by the fact that is in the treatment of chronic-degenerative diseases comprised in the group of rheumatoid arthritis, osteoarthritis, systemic lupus eritematosus, ulcerative colitis, psoriasis, atopic eczema, atherosclerosis, or in the treatment of non degenerative diseases comprised in the group of depression, and cellulites, and allergies.

6. Use of caryophyllenes according to one of claims 1-4 characterized by the fact that it is in the manufacture of a medicament for the treatment of chronic-degenerative diseases comprised in the group of rheumatoid arthritis, osteoarthritis, systemic lupus eritematosus, ulcerative colitis, psoriasis, atopic eczema, atherosclerosis, or in the treatment of non degenerative diseases comprised in the group of depression, and cellulites, or allergies.

7. Use of caryophyllenes, or compositions comprising caryophyllenes, according to one of claims 1 to 4, characterized by the fact that it is administered via enteral or parenteral, including oral, topical, transdermal, subcutaneous, intraperitonial, intravenous, by infiltration, by inhalation, transdermal, transmucosal, intramuscular, intrapulmonary, vaginal, rectal, intraocular, and sublingual.

8. Use according to claim 7, characterized that said administration is topical or systemical, particularly chosen among infiltration, oral, inhalation or transdermal.

9. Use of caryophyllenes, or compositions comprising caryophyllenes, according to one of claims 1 to 4 characterized by the fact that said caryophyllenes are one or more of alpha-humulene and trans-caryophyllene.

10. A method of treatment of inflammatory conditions and inflammatory pain of the animal body, particularly the human body, characterized by the fact that it comprises the administration of a therapeutically effective amount of caryophyllenes, to a patient.

11. A method according to claim 10 characterized by the fact that said inflammatory conditions and inflammatory pain are present in chronic-degenerative diseases comprised in the group of rheumatoid arthritis, osteoarthritis, systemic lupus eritematosus, ulcerative colitis, psoriasis, atopic eczema, atherosclerosis, or in the non degenerative diseases comprised in the group of depression, and cellulites, or in allergies.

12. A method of inhibiting the bodily production of one or more of cytokine IL-1β, cytokine TNFα, prostaglandin PGE2, or expression of enzymes COX-2 and iNOS, characterized by the fact that it comprises the administration of a therapeutically effective amount of caryophyllenes, to a patient.

13. A method according to one of claims 10 to 12 characterized by the fact that said caryophyllene is one or more of alpha-humulene and trans-caryophyllene.

14. A composition comprising caryophyllene characterized by the fact that the amount of said caryophyllene is about 1 to about 1000 mg.

15. A composition according to claim 14 characterized by the fact that said amount is about 10 to about 200 mg.

16. A composition according to claim 14 characterized by the fact that said amount is about 30 to about 100 mg.

17. A composition according to claim 14 characterized by the fact that said caryophyllene comprises about 0.1 to about 99% in weight of said composition.

18. A composition according to claim 14 characterized by the fact that said caryophyllene comprises about 1 to about 70% in weight of said composition.

19. A composition according to claim 14 characterized by the fact that said caryophyllene comprises about 10 to about 40% in weight of said composition.

20. A composition according to one of claims 15 to 19 characterized by the fact that said caryophyllene is one or more of alpha-humulene and trans-caryophyllene.
